(19) United States
(12) Patent Application Publication
(10) Pub. No.: US 2025/0057151 A1
(43) Pub. Date: Feb. 20, 2025

(54) PACKAGING FOR PRESERVATION OF BIOLOGICAL MATERIAL

(71) Applicant: VITRAFY LIFE SCIENCES LIMITED, Hobart (AU)

(72) Inventors: Brent Owens, Hobart (AU); Sean Cameron, Hobart (AU)

(21) Appl. No.: 18/717,672

(22) PCT Filed: Dec. 22, 2022
(86) PCT No.: PCT/AU2022/051570

§ 371 Date: Jun. 7, 2024

(30) Foreign Application Priority Data
Dec. 23, 2021 (AU) 2021904254

(51) Int. Cl. A01N 1/02

(52) U.S. Cl. CPC A01N 1/0263; A01N 1/0284

(57) ABSTRACT

Packaging for preservation of biological material, wherein, in use, the packaging is filled with biological material and placed in an apparatus for preserving the biological material such that a heat exchange fluid flows around the packaging, the packaging including: one or more packaging walls configured to define an internal compartment for receiving the biological material; and one or more thermal contours defined across at least one of the packaging walls, wherein, in use, the flow of the heat exchange fluid is at least partially directed by the one or more thermal contours to improve heat transfer between the heat exchange fluid and the biological material contained in the packaging.

### BACKGROUND OF THE INVENTION

The present invention relates to packaging for preservation of biological material, being particularly, but not exclusively, suitable for cryopreservation, cryostorage and thawing of biological material such as whole blood and the like.

### DESCRIPTION OF THE PRIOR ART

The ability to store red blood cells (RBCs) outside of the body has been regarded as a life-saving practice for many years. More recently, the usage of refrigerated stored RBCs in transfusion medicine has been under extensive evaluation. During refrigerated storage RBCs progressively deteriorate and infusion of prolonged stored RBCs has been linked to adverse clinical outcome in terms of postoperative infections, length of hospital stay and mortality.

RBCs are subject to international guidelines requiring that haemolysis in the RBC units must remain below allowable levels (i.e.. 0.8% in Europe and 1% in The United States) and that the RBC post-thaw recovery after deglycerolisation (i.e. freeze-thaw-wash recovery) must exceed 80%. Also, at least 75% of cryopreserved RBCs must still circulate 24 hours after infusion.

Freezing large quantities of cells is a necessity for therapeutic cell banking. This requires either large volumes or reduced volumes of highly concentrated cells. Additionally, bioreactor systems often require seeding with high numbers of cells from working cell banks or starter cultures.

Medical device packaging is almost as important as the device itself. Packaging for medical devices plays a key role in safely delivering specialized treatment to patients. Most single use, sterilized medical devices can be opened with a high degree of confidence that it has remained sterile throughout storage, handling, and transportation.

What makes medical device packaging doubly important is that regulatory authorities recognize the critical nature of sterile barrier or primary package by considering them components or accessories to the medical device. This implies that packaging is almost as important as the device itself.

The rate at which a biospecimen cools has been proven to have a dramatic impact on its long-term viability. Not only does cooling rate affect the rate of formation and size of both intracellular and extracellular ice crystals; it also can impact solution effects that occur during the freezing process.

Crystallization occurs through ice crystal nucleation and growth in both intracellular and extracellular regions when the temperature of the liquid approaches its crystallization temperature. The crystallization is the phase transition of the first order including energy release owing to the latent heat of fusion. A number of factors such as cooling rate, homogeneity and pressure affect the phase transition from liquid to solid.

The cooling rate is another parameter that has been used to optimize cell viability (Dumont, F., P. A. Marechal, and P. Gervais. 2003. Influence of cooling rate on Saccharomyces cerevisiae destruction during freezing: unexpected viability at ultra-rapid cooling rates. Cryobiology 46:33-42.). Simultaneous management of cryoprotective agents (CPAs) and the cooling rate, could be used. Indeed, the cooling rate determines ice crystal size during freezing. As a solution begins to freeze, water in the extracellular fluid turns to ice and hence increases the solute concentration in the liquid outside the cells. Subsequent osmosis then dehydrates cells as water diffuses from the cytoplasm into the more concentrated external solution (Dumont, F., P. A. Marechal, and P. Gervais. 2004. Cell size and water permeability as determining factors for cell viability after freezing at different cooling rates. Appl. Environ. Microbiol. 70:268-272.). When cooling rates are low, osmotically driven flow results in a reduction in volume, and all of the intracellular water can flow out before intracellular crystallization. When the cooling rates are in the middle range, the reduction in volume leads to irreversible damage to the cell, and significant mortality is observed. When cooling rates are very high, the cell may not have time to reduce its volume because of the rapid heat flow, and this may allow maintenance of significant viability. Therefore, the kinetics of freezing have a great effect on cell viability; however, most of the time the cooling rate is not well controlled.

Cell death occurs due to the massive water outflow during slow cooling, which was related to an increase in the extracellular osmotic pressure and the membrane-lipid phase transition, or from crystallization during water outflow from the cell, which involved lethal membrane damage.

Based on this assumption, cell death occurs during the lag time determined by the crystallization of the medium. Cell mortality corresponds to temperatures between 0° C. and −5° C. However, there was a second stage of cell mortality at lower temperatures.

For solutions mainly composed of water, the range of temperatures at which almost all crystallization takes place is reduced to a few degrees after the onset of freezing, and the crystallization temperature is dependent on the concentration of the solute.

AU2009258341B2 discloses a packaging for biological material, comprising two substantially parallel walls connected to each other along a part of their periphery and in a central area of the packaging. The central area, wall area and thickness, and periphery are configured to ensure that the walls have sufficient stiffness to stay parallel after filling the packaging with biological material. The packaging may be used for cryopreservation of biological material.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

### SUMMARY OF THE PRESENT INVENTION

In one broad form, an aspect of the present invention seeks to provide packaging for preservation of biological material, wherein, in use, the packaging is filled with biological material and placed in an apparatus for preserving the biological material such that a heat exchange fluid flows around the packaging, the packaging including: one or more packaging walls configured to define an internal compartment for receiving the biological material; and one or more thermal contours defined across at least one of the packaging walls, wherein, in use, the flow of the heat exchange fluid is at least partially directed by the one or more thermal contours to improve heat transfer between the heat exchange fluid and the biological material contained in the packaging.

In one embodiment, the thermal contours are arranged to substantially align with the flow of the heat exchange fluid in use.

In one embodiment, the packaging includes a plurality of thermal contours that are arranged to be parallel to one another.

In one embodiment, each of the thermal contours is defined across a respective one of the packaging walls as one of: an elongate depression in the respective packaging wall; and an elongate protrusion in the respective packaging wall.

In one embodiment, the packaging walls include opposing first and second walls, the first and second walls being connected together around a substantial portion of their respective perimeters to define the internal compartment.

In one embodiment, the one or more thermal contours are defined by the first and second walls being connected together along one or more connection lines, the connection lines being configured to divide the internal compartment into sub-compartments with fluid communication allowed therebetween.

In one embodiment, in use, the packaging is filled with biological material so that the biological material is distributed between the sub-compartments, and the flow of the heat exchange fluid is at least partially directed by the one or more thermal contours to facilitate substantially even heat transfer between the heat exchange fluid and the biological material contained in each of the sub-compartments.

In one embodiment, wherein the connection lines are configured to ensure that the biological material is distributed substantially evenly between each of the sub-compartments.

In one embodiment, the first and second walls are connected together along a plurality of edges including a leading edge that faces the flow of heat transfer fluid in use, and an opposing trailing edge, the plurality of thermal contours extending between the leading edge and the trailing edge.

In one embodiment, the trailing edge is substantially parallel to the leading edge.

In one embodiment, at least some of the connection lines interconnect with the leading edge.

In one embodiment, the thermal contours are arranged at a predetermined angle relative to a direction perpendicular from the leading edge.

In one embodiment, the predetermined angle is selected according to the flow of heat exchange fluid in the apparatus for preserving the biological material.

In one embodiment, the predetermined angle is at least one of: between 0° and 30°; between 5° and 15°; and about 10°.

In one embodiment, adjacent thermal contours are spaced apart by a predetermined spacing distance.

In one embodiment, the predetermined spacing distance is between 15 mm and 20 mm.

In one embodiment, the predetermined spacing distance is selected to restrict separation of the first and second walls.

In one embodiment, the predetermined spacing distance is selected to restrict separation of the first and second walls to a predetermined separation distance.

In one embodiment, a packaging depth measured between the first and second walls is at least one of: less than 10 mm; less than 5 mm; between 1 mm and 4 mm; and less than 1 mm.

In one embodiment, the packaging is configured so that the first and second walls remain substantially parallel to one another when the packaging is filled with the biological material in use.

In one embodiment, the first and second walls are formed from sheets of a packaging material.

In one embodiment, the packaging material is selected from one of: polymers; polypropylene; polyvinyl chloride; polyethylene terephthalate; ethylene vinyl acetate copolymer; copolymers; ethylene and vinyl acetate; metals; high alloy; and stainless steel.

In one embodiment, the packaging includes one or more openings for facilitating filling and emptying of the packaging.

In one embodiment, the one or more openings include one or more ports extending through an edge of the packaging.

In one embodiment, the packaging is configured for preservation of biological material that is selected from one of: whole blood; blood platelets; red blood cells; white blood cells; plasma; blood products; sperm; cells; stem cells; organs or parts thereof; and tissue.

In one embodiment, the packaging is configured for preservation of biological material to be used for therapeutic treatments.

In one embodiment, the packaging is configured for at least one of: cryopreservation of biological material; cryostorage of biological material; and thawing of biological material.

In one embodiment, the packaging is configured for use with a heat transfer rate selected from one of: between 0° C. and 10° C. per minute; between 10° C. and 50° C. per minute; between 50° C. and 100° C. per minute; and greater than 100° C. per minute.

In one embodiment, the packaging is configured as a bag.

In one embodiment, the packaging is configured as one of: a straw; and a vial.

In another broad form, an aspect of the present invention seeks to provide packaging for preservation of biological material, wherein, in use, the packaging is filled with biological material and placed in an apparatus for preserving the biological material such that a heat exchange fluid flows around the packaging, the packaging including: opposing first and second walls, the first and second walls being connected together around a substantial portion of their respective perimeters to define an internal compartment; and a plurality of thermal contours defined by the first and second walls being connected together along connection lines, the connection lines being configured to divide the internal compartment into sub-compartments with fluid communication allowed therebetween, wherein, in use, the biological material is distributed between the sub-compartments, and the flow of heat exchange fluid is at least partially directed by the thermal contours to improve heat transfer between the heat exchange fluid and the biological material contained in the sub-compartments of the packaging.

In another broad form, an aspect of the present invention seeks to provide a method for use in designing packaging for preservation of biological material, wherein, in use, the packaging is filled with biological material and placed in an apparatus for preserving the biological material such that a heat exchange fluid flows around the packaging, the method including: a) determining a packaging geometry including one or more packaging walls configured to define an internal compartment to allow the packaging to be filled with a desired volume of the biological material; b) determining thermal properties of: the biological material; packaging material for forming the one or more packaging walls; and the heat exchange fluid; c) determining operating conditions of the apparatus including: velocity of the heat exchange fluid; temperature of the heat exchange fluid; and flow direction of the heat exchange fluid; d) perform analysis on the flow of the heat exchange fluid within the apparatus around the filled packaging, in accordance with the determined packaging geometry, thermal properties and operating conditions, to determine expected temperature gradients in the biological material in use; e) using the expected temperature gradients to select a configuration of one or more thermal contours defined across at least one of the packaging walls to improve heat transfer between the heat exchange fluid and the biological material contained in the packaging; and f) performing further analysis on the flow of the heat exchange fluid within the apparatus around the filled packaging, including the of one or more thermal contours, in accordance with the determined packaging geometry, the selected configuration of the one or more thermal contours, thermal properties and operating conditions, to determine expected temperature gradients in the biological material in use.

In one embodiment, the method includes repeating steps e) and f) until desirable expected temperature gradients are determined.

In one embodiment, the packaging geometry includes opposing first and second walls, the first and second walls being connected together around a substantial portion of their respective perimeters to define the internal compartment, and a plurality of thermal contours defined by the first and second walls being connected together along connection lines, the connection lines being configured to divide the internal compartment into sub-compartments with fluid communication allowed therebetween, the method including: performing the analysis on the flow of the heat exchange fluid within the apparatus around the filled packaging; and using the expected temperature gradients to select a configuration of the thermal contours to provide substantially even heat transfer between the heat exchange fluid and the biological material contained in each of the sub-compartments.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:—

FIG. 1 is a plan view of an example of packaging for preservation of biological material;

FIG. 2A is a schematic plan view of the packaging of FIG. 1;

FIG. 2B is a schematic side view of the packaging of FIG. 2A;

FIG. 2C is a schematic cross section view of the packaging of FIG. 2A at section A-A;

FIG. 3A is a schematic plan view of a simplified analysis model of the packaging of FIG. 2A, showing analysis positions across the packaging;

FIG. 3B is a schematic cross section view of the simplified analysis model of the packaging of FIG. 3A at section A-A;

FIG. 3C is a schematic detail view of the cross section view of the simplified analysis model of the packaging of FIG. 3B at detail B, showing analysis points within the packaging;

FIG. 4A is a plot of expected temperature gradients over time at analysis position P101 as shown in FIG. 3A, during cryopreservation of whole blood using the packaging;

FIG. 4B is a plot of expected temperature gradients over time at analysis position P103 as shown in FIG. 3A, during cryopreservation of whole blood using the packaging;

FIG. 4C is a plot of expected temperature gradients over time at analysis position P105 as shown in FIG. 3A, during cryopreservation of whole blood using the packaging;

FIGS. 5A to 5D show temperature maps for a central layer of the packaging at subsequent time intervals, during cryopreservation of whole blood using the packaging;

FIGS. 6A to 6D show temperature maps at subsequent time intervals, during cryopreservation of whole blood using a conventional blood bag;

FIGS. 7A and 7B show top and side views of computational fluid dynamics (CFD) analysis results depicting a flow of heat transfer fluid in an apparatus for preservation of biological material, for use with the packaging; and

FIGS. 8A and 8B show plots depicting relationships between bag width, blood volume and RBC viability.

### DETAILED DESCRIPTION OF THE PREFERRED EMBODIMENTS

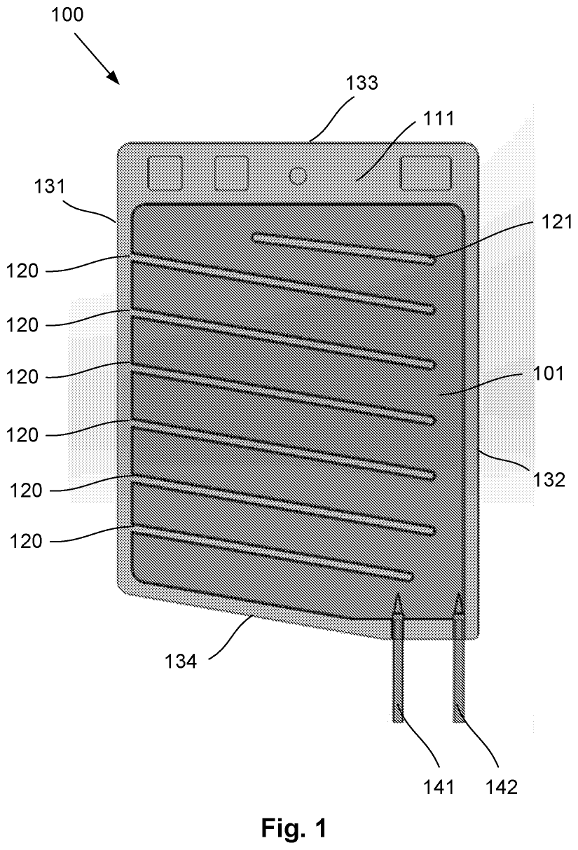

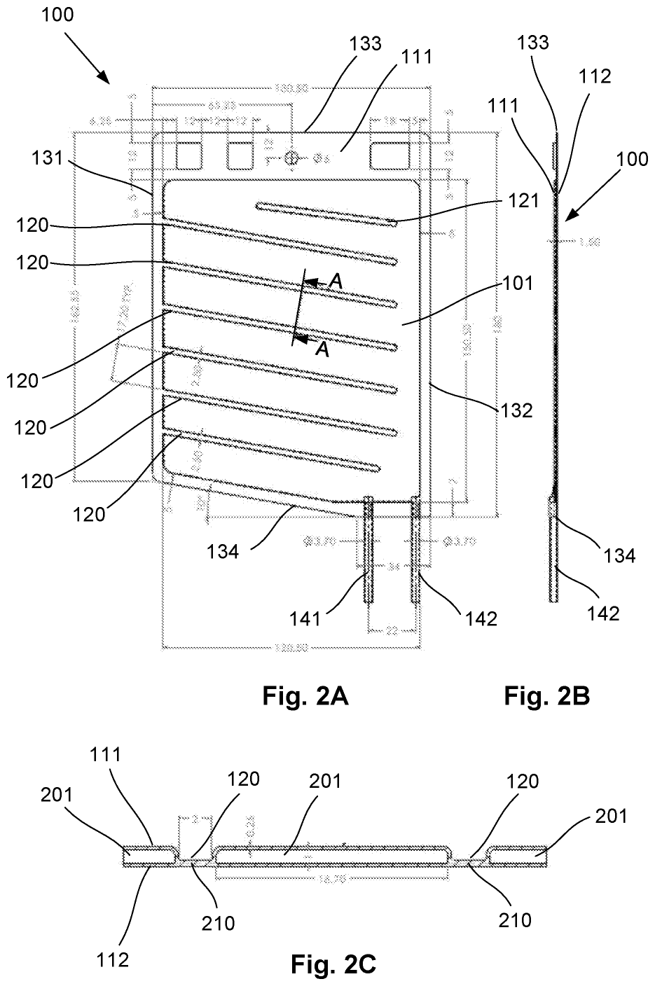

An example of packaging 100 for preservation of biological material will now be described with reference to FIG. 1 and FIGS. 2A to 2C. The packaging 100 is provided so that, in use, the packaging 100 may be filled with biological material and placed in an apparatus for preserving the biological material such that a heat exchange fluid flows around the packaging 100.

As used herein, “biological material” includes the following non-exhaustive list of materials: blood, plasma, platelets, leucocytes or other blood products; germs, viruses bacteria, fungi, or other microorganisms; organs or parts thereof, seminal fluid, eggs, colostrum, skin, serum, vaccines, stem cells (e.g. from bone marrow, umbilical cord blood, amniotic fluid, etc.), umbilical cords, bone marrow, germ cells, tumour cells, colostrum, and plant cells.

Embodiments of the packaging 100 described herein are specifically configured for preservation of whole blood as the biological material, although it should be appreciated that the packaging 100 may be configured for a wide range of biological materials including other materials that are not explicitly mentioned herein.

As used herein, “preservation” of biological material refers to a variety of processes that may be used in connection with the storage of biological material. In some examples, preservation may involve freezing or cooling the biological material, storage of the frozen or cooled biological material, and thawing the biological material or otherwise returning the biological material to a desired temperature for subsequent use as required. Preferred embodiments of the packaging may be specifically configured for at least one of cryopreservation of biological material, cryostorage of biological material, and thawing of biological material.

Examples of suitable apparatus for preserving the biological material are described in WO2020/102854A1, the entire disclosures of which are incorporated herein by reference. For instance, as described in the aforementioned publication, the apparatus may comprise an inner housing arranged within an outer insulated housing, wherein walls of the inner housing define a compartment for receiving biological products, said walls comprising an inlet wall for inflow of a heat exchange fluid into the compartment, an opposed outlet wall for outflow of a heat exchange fluid out of the compartment, side walls and a base, the side walls and base adjoining the inlet wall to the outlet wall, wherein the inlet wall and outlet wall each include a series of apertures to accommodate a continuous heat exchange fluid flow through the apparatus such that, in operation, an item received in the compartment of the inner housing are immersed in the heat exchange fluid to exchange heat with the heat exchange fluid. In this context, the packaging 100 is filled with biological material and placed in the compartment of the inner housing of the apparatus such that a heat exchange fluid flows around the packaging 100.

In broad terms, the packaging 100 includes one or more packaging walls 111, 112 configured to define an internal compartment 101 for receiving the biological material, and one or more thermal contours 120, 121 defined across at least one of the packaging walls 111. In use, the flow of the heat exchange fluid is at least partially directed by the one or more thermal contours 120, 121 to improve heat transfer between the heat exchange fluid and the biological material contained in the packaging 100.

For example, as the heat exchange fluid flows around the packaging 100 within the apparatus, the thermal contours 120, 121 may help to direct the flow of the heat exchange fluid across the respecting packaging wall to thereby facilitate more even heat transfer between the heat exchange fluid and the biological material. This could help to avoid regions of relatively higher or lower heat transfer which could otherwise lead to the presence of “hot spots” or “cold spots” in the biological material in use. It will be appreciated that such hot spots or cold spots are generally undesirable as these represent variations in the heat transfer rate which could have adverse impacts upon cell viability or the like during or after the preservation of the biological material.

In contrast, it should be understood that the thermal contours 120, 121 may be provided to at least partially control or optimise the heat transfer rate between the heat exchange fluid and the biological material. For instance, the thermal contours 120, 121 may be configured with regard to the packaging geometry, thermal properties of the biological material, packaging material and the heat exchange fluid, and operating conditions of the apparatus, to ensure that the heat transfer between the heat exchange fluid and the biological material is substantially evenly distributed relative to the packaging geometry. In this regard, the configuration of the thermal contours 120, 121 may be selected based on a thermal analysis of the packaging in its intended use, and suitable techniques for doing so will be discussed in further detail in due course.

In any event, it will be appreciated that the use of packaging 100 in which thermal contours 120, 121 are defined across at least one of the packaging walls 111 can facilitate improved cryopreservation, cryostorage and/or thawing of the biological material compared to the use of traditional packaging without any thermal contours 120, 121.

Further details of preferred and/or optional features of suitable embodiments of the packaging 100 will now be described with regard to FIG. 1 and FIGS. 2A to 2C.

In preferred examples, the thermal contours 120, 121 may be arranged to substantially align with the flow of the heat exchange fluid in use. For example, the expected flow direction of the heat exchange fluid within the apparatus and relative to the packaging 100 in use may be determined experimentally or theoretically, for instance by performing a thermal analysis of the flow conditions, such as by using computational fluid dynamics (CFD) analysis or the like. Thus, the packaging may be configured so that the thermal contours 120, 121 are substantially aligned with the expected flow direction. This may involve, for example, arranging the thermal contours 120, 121 at a predetermined angle relative to the packaging geometry, as will be discussed in further detail below.

In many embodiments, the packaging 100 may include a plurality of thermal contours 120, 121 that are preferably arranged to be parallel to one another. It will be appreciated that such a parallel arrangement may help to improve how the thermal contours 120, 121 direct the flow of the heat exchange fluid relative to the packaging 100 in use. However, it should be understood that it is not essential to provide a plurality of thermal contours and in some examples a single thermal contour may be provided. For instance, in some packaging geometries, such as a straw or tube configuration, a single thermal contour may be provided in a spiral arrangement across a cylindrical wall of the packaging 100.

In the example embodiment as shown in FIG. 1 and FIGS. 2A to 2C, each of the thermal contours 120, 121 may be defined across a respective one of the packaging walls 111 as an elongate depression in the respective packaging wall 111, as best observed in the cross section profile shown in FIG. 2C. In this example, each of the thermal contours 120, 121 may be described as a groove, channel or recess in the respective packaging wall 111. However, in alternative embodiments, the thermal contours 120, 121 may be defined as an elongate protrusion in the respective packaging wall 111, for example in the form of a ridge or the like in the respective packaging wall. In either case, it will be appreciated that the thermal contours 120, 121 will assist in directing the flow of the heat transfer fluid in use.

In some embodiments, such as that shown in FIG. 1 and FIGS. 2A to 2C, the packaging walls 111, 112 include opposing first and second walls, the first wall 111 and the second wall 112 being connected together around a substantial portion of their respective perimeters to define the internal compartment 101. In this context, the thermal contours 120, 121 may be defined by the first wall 111 and the second wall 112 being connected together along one or more connection lines 210. For instance, as shown in FIG. 2C, the thermal contours 120 may be formed as elongate depressions in the first wall 111, where the first wall 111 is connected to the second wall 112 along respective connection lines 210.

The connection lines 210 may be configured to effectively divide the internal compartment 101 into sub-compartments 201 with fluid communication allowed therebetween. In use, the packaging 100 may be filled with biological material so that the biological material is distributed between the sub-compartments 201, and the flow of the heat exchange fluid will be at least partially directed by the thermal contours 120, 121 to facilitate substantially even heat transfer between the heat exchange fluid and the biological material contained in each of the sub-compartments 201. In preferred examples, the connection lines 210 may be configured to ensure that the biological material is distributed substantially evenly between each of the sub-compartments 201.

It will be appreciated that the combination of this even distribution of the biological material between the sub-compartments 201, together with the thermal contours 120, 121 directing the flow of the heat exchange fluid to facilitate substantially even heat transfer between the heat exchange fluid and the biological material contained in each of the sub-compartments 201, can help to minimise temperature variations throughout the biological material and thereby allow for consistent heat transfer during preservation of the biological material.

In some embodiments, the first wall 111 and the second wall 112 may be connected together along a plurality of edges including a leading edge 131 that faces the flow of heat transfer fluid in use, and an opposing trailing edge 132. The plurality of thermal contours 120, 121 will preferably extend between the leading edge 131 and the trailing edge 132. It will be understood that the thermal contours 120, 121 will direct the flow of the heat exchange fluid as it reaches the leading edge 131 and subsequently across the respective wall of the packaging 100 towards the trailing edge 132.

Typically the trailing edge 132 is substantially parallel to the leading edge 131, such as in in the packaging 100 configuration shown in FIG. 1 and FIGS. 2A to 2C, but this is not necessarily always the case.

At least some of the connection lines 210, which define the thermal contours 120, 121, may interconnect with the leading edge 131. Such an arrangement may assist in providing the packaging with a more streamlined profiled at the leading edge 131 so as to prevent disruption of the flow of the heat transfer fluid around the leading edge 131.

However, it should be appreciated that not all of the connection lines 210 and associated thermal contours 120, 121 will necessarily interconnect with the leading edge 131. In this regard, it is noted that one of the thermal contours 121 in the example of FIG. 1 and FIGS. 2A to 2C is not interconnected with the leading edge 131. It will be understood that interconnecting this thermal contour 121 to the leading edge 131 would create a small sub-compartment that might trap biological material in a corner thereof, which may be undesirable for heat transfer and may also inhibit filling or emptying of the packaging 100.

It should also be appreciated that the thermal contours 120, 121 may extend all the way to the leading edge 131, in a similar manner to the arrangement above, even in embodiments that do not include connection lines 210, 211.

The thermal contours 120, 121 may be arranged at a predetermined angle relative to a direction perpendicular from the leading edge 131. Preferably, the predetermined angle is selected according to the flow of the heat exchange fluid in the apparatus for preserving the biological material. For instance, the predetermined angle may be selected so as to substantially align the thermal contours 120, 121 with the flow of the heat exchange fluid as mentioned above.

In some examples, the predetermined angle may be between 0° and 30°. Preferably, the predetermined angle may be between 5° and 15°. In the particular embodiment of FIGS. 2A to 2C, the predetermined angle may be about 10°. However, it should be understood that the predetermined angle may be selected outside of these ranges, for example if the direction of the flow of the heat exchange fluid is at a steeper angle than mentioned above.

In examples of the packaging 100 including a plurality of thermal contours 120, 121, the adjacent thermal contours 120, 121 will typically be spaced apart by a predetermined spacing distance. For example, in the embodiment of FIGS. 2A to 2C, the predetermined spacing distance may be between 15 mm and 20 mm. However, the particular spacing distance will typically depend on the geometrical configuration of the packaging 100 and may also depend on other factors such as the particular biological material the packaging 100 will be filled with.

As a general principle, the predetermined spacing distance may be selected to restrict separation of the first wall 111 and the second wall 112, particularly in use. It will be understood that, in embodiments in which the thermal contours 120, 121 are defined so as to coincide with connection lines 210, the first wall 111 and the second wall 112 will be prevented from separating at the connection lines 210, and the separation between the first wall 111 and the second wall 112, such as due to bulging when the packaging is filled with the biological material, will be restricted depending on the predetermined spacing distance between the adjacent thermal contours 120, 121.

In preferred embodiments, the predetermined spacing distance may be selected to restrict separation of the first wall 111 and the second wall 112 to a predetermined separation distance. It will be appreciated that a relatively smaller predetermined separation distance may be provided by reducing the predetermined spacing distance and that a relatively larger predetermined separation distance may be allowed by increasing the predetermined spacing distance.

In some examples, the packaging depth measured between the first wall 111 and the second wall 112 will preferably be less than 10 mm. The packaging depth may be less than 5 mm, and in some embodiments the packaging depth may be between 1 mm and 4 mm. However, in some applications it may be desirable to provide a smaller packaging depth, such as less than 1 mm. It should be appreciated that the optimal packaging depth will typically depend on the overall packaging geometry and thermal considerations, some of which will be described in further detail in due course.

Preferably, the packaging 100 will be configured so that the first and second walls 111, 112 remain substantially parallel to one another when the packaging 100 is filled with the biological material in use. This may be achieved through a combination of the spacing between connection lines 210 and associated thermal contours 120, 121

As far as the construction of the packaging 100 is concerned, the first and second walls 111, 112 may be formed from sheets of a packaging material. For example, the packaging material may be selected from polymers (such as polypropylene, polyvinyl chloride, polyethylene terephthalate, or ethylene vinyl acetate copolymer), copolymers (such as ethylene and vinyl acetate) or metals (such as high alloy metals or stainless steel. It will be understood that different packaging materials may allow packaging with different properties.

For instance if the first and second walls 111, 112 are formed from sheets of flexible material, such as polymers, this may result in packaging that is subject to bulging when filled, but this can be controlled, for example, by selecting appropriate spacing distances between the thermal contours 120, 121 as discussed above. The connections between the first and second walls 111, 112 may be achieved by heat sealing or other suitable thermo-forming techniques.

On the other hand, if the first and second walls 111, 112 are formed from sheets of rigid material, such as metals, the thermal contours 120, 121 may be defined as deformations of the sheets without requiring connections between the first and second walls 111, 112 or particular spacing distances therebetween. However, connections between the first and second walls 111, 112 may nevertheless be provided such as by welding, so as to define sub-compartments for the reasons discussed above.

The packaging 100 may include one or more openings for facilitating filling and emptying of the packaging 100. These openings may include one or more ports 141, 142 extending through an edge 134 of the packaging 100, as shown in FIGS. 2A and 2B. Preferably the ports 141, 142 will be provided on an edge 134 other than the leading edge 131 and the trailing edge 132. In this example, the ports 141, 142 may extend away from the first and second walls 111, 112 of the packaging 100. In one specific example each port 141, 142 may include a Luer lock connector for facilitating connections to tubing or the like to aid filling or emptying of the packaging.

The packaging may be configured for preservation of a range of different biological materials, such as biological materials selected from one of: whole blood; blood platelets; red blood cells; white blood cells; plasma; blood products; sperm; cells; stem cells; organs or parts thereof; and tissue.

As mentioned above, the example embodiment of the packaging 100 as shown in FIG. 1 and FIGS. 2A to 2C has been particularly configured for use in the cryopreservation of whole blood.

In other embodiments, the packaging may be particularly configured for use in the preservation of stem cells or the like, such as: adult stem cells such as hematopoietic stem cells, mesenchymal stem cells, neural stem cells, epithelial stem cells, skin stem cells; embryonic/pluripotent stem cells; induced pluripotent stem cells; bone marrow derived stem cells; or umbilical cord blood.

In yet other embodiments, the packaging may be configured for use in the preservation of a range of other types of biological materials, such as: chimeric antigen receptor T (CAR-T) cells, genetically modified cells, natural killer cells, blastocytes, embryos, oocytes, zygote, ovarian tissue, testicular tissue, sperm, and heart valves.

It should be appreciated that the examples of biological materials identified herein are not intended to be an exhaustive list, and the packaging may also be used in the preservation of other biological materials.

In some examples, the packaging 100 may be specifically configured for preservation of biological material to be used for therapeutic treatments, such as CAR-T cell therapy.

The packaging 100 may be configured for use with a range of different apparatus for preserving the biological material, with a range of different operational parameters. For example, the packaging 100 may be configured for compatibility with a heat transfer rate selected from: between 0° C. and 10° C. per minute; between 10° C. and 50° C. per minute; between 50° C. and 100° C. per minute; and greater than 100° C. per minute.

The packaging 100 can also be provided in a range of different packaging geometries and form factors whilst providing functionality as discussed above. For instance, the packaging 100 may be configured as a bag, as shown in FIG. 1 and FIGS. 2A to 2C. However, the packaging may alternatively be configured as a straw or a vial in other examples. It will also be appreciated that features of the present invention may be applied to packaging in a variety of other form factors.

In one preferred form, the packaging 100 may be specifically configured to include: opposing first and second walls 111, 112, the first and second walls 111, 112 being connected together around a substantial portion of their respective perimeters to define an internal compartment 101; and a plurality of thermal contours 120, 121 defined by the first and second walls 111, 112 being connected together along connection lines 210. The connection lines 210 are configured to divide the internal compartment 101 into sub-compartments 201 with fluid communication allowed therebetween. In use, the biological material is distributed between the sub-compartments 201, and the flow of heat exchange fluid is at least partially directed by the thermal contours 120, 121 to improve heat transfer between the heat exchange fluid and the biological material contained in the sub-compartments 201 of the packaging.

Further detailed design considerations that may be applied in configuring preferred embodiments of the packaging 100 will now be described.

In designing embodiments of the packaging 100, and as a result of validation testing of these embodiments, the applicant encountered problems associated with the uniformity of heat transfer across the packaging 100 in use. There was a study conducted which found a correlation between varying packaging dimensions, configurations and materials and decreasing cell viability. This study specifically focused on the design and manufacture of packaging bags for the cryopreservation of blood and blood products. Validation testing confirmed that the more uniformed heat transfer remains along the entirety of the bag, cell viability would remain the same. Furthermore, by conducting thermal analysis and including thermal contours as part of the packaging as described above, heat transfer coefficients were improved for a more controlled heat transfer as volumes of the packaging increased.

The specific configuration of the packaging 100 as shown in the embodiment of FIG. 1A and FIGS. 2A to 2C was determined as a result of this study and thermal analysis, further details of which will now be outlined.

The packaging 100 is specifically provided in the form of a packaging bag configured for use in the cryopreservation of whole blood, and has been designed to have a 15 mL volume with overall dimensions of approximately 150 mm×120 mm.

In this embodiment, a plurality of thermal contours 120, 121 were included to direct heat exchange fluid flow around each sub-compartment defined between them, to allow for even heat transfer between the heat exchange fluid.

Spacing requirements between the thermal contours 120, 121 was determined by overall minimum sample volume. The spacing between thermal contours 120, 121 was used to create substantially evenly distributed sub-compartments across the overall packaging bag to allow for uniformed heat transfer. The spacing between adjacent thermal contours 120, 121 is a minimum of 15 mm and a maximum of 20 mm. As the packaging bag volume and dimensions increase, the same minimum and maximum spacing may still be used.

The thermal contours 120, 121 have been arranged to align with the directional flow of heat exchange fluid within a cryopreservation apparatus in use, to allow for even and uniformed heat transfer.

It has been determined that raised edges of the packaging can disrupt directional flow of heat exchange fluid in use. To eliminate this problem and improve the consistency in heat transfer, the thermal contours 120, 121 were taken to the leading edge 131 of the packaging bag to seek to reduce bulging of the packaging in this region and thus seek to reduce disruption in the flow of the heat exchange fluid.

A total of seven thermal contours 120, 121 have been included for this particular design. As sample and bag volumes increase, it is expected that additional thermal contours 120, 121 may be included to provide additional, evenly distributed sub-compartments.

In the embodiment of FIG. 1 and FIGS. 2A to 2C, the packaging 100 is configured as a clam-shell semi-rigid design using 250-micron, food and medical grade glycol modified polyethylene terephthalate (PETG), which was chosen for its suitability for lower temperature thermo-forming, and low temperature scaling (as approximately 140° C.). The packaging 100 includes a formed 2-port design for filling/emptying and venting.

The standard method for filling IV Bags in medical industry is typically using a Luer lock, which may also be employed in the port design. These are a standard in terms of thread, taper and seal to achieve a clean, safe, sterile filling point. Furthermore, the ports may use EVA tubing which lends itself to a range of ultrasonics for scaling.

The packaging 100 may be manufactured utilizing a clam-shell type manufacturing method involving forming 2 sheets of 250-micron PETG plastic to manufacture a closed volume vessel. In broad terms, the manufacturing method included steps of: mold design and 3D print, vacuum forming the 0.25 mm PETG sheets; trimming the parts; welding the parts using a constant heat scaler; and performing pressure testing/quality checking.

The packaging 100 was designed using 3D design software. In this regard, 3D computer aided design (CAD) models were developed in three different configurations, such that each step of the design process could be facilitated, as follows:
Configuration 1: mold shape for 3D printing, used for subsequent vacuum forming process;
Configuration 2: simplified analysis model for computational fluid dynamics (CFD) analysis of the heat transfer; and
Configuration 3: finished part 3D representation of the packaging.

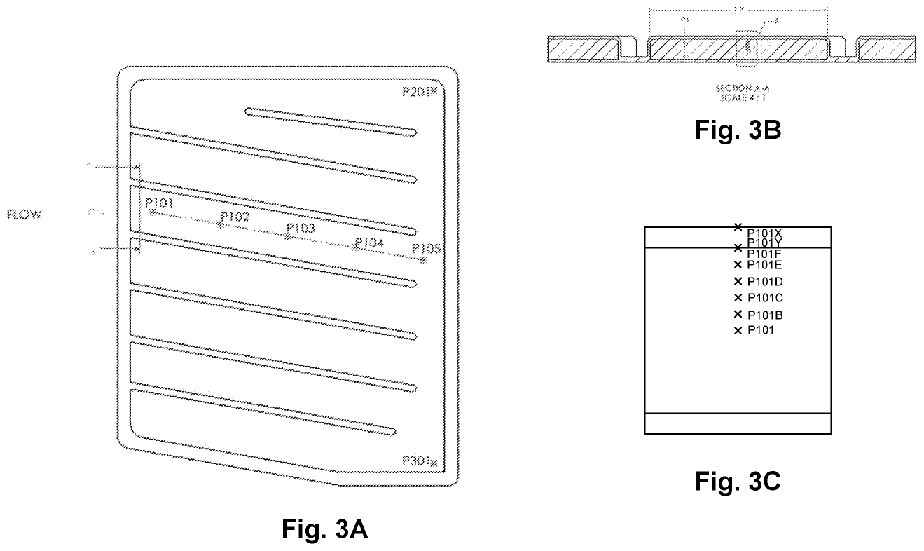

The analysis model used for the CFD analysis is shown in FIGS. 3A to 3C. Using this analysis model, temperature gradients were analysed from the core through to the package skin at various locations as indicated in FIGS. 3A to 3C. FIG. 3A shows analysis positions across the packaging, and particularly analysis positions P101, P102, P103, P104 and P105 which are spaced apart in the same sub-compartment between thermal contours, in the flow direction of the heat exchange fluid as shown.

FIG. 3B shows a cross section view of the analysis model of FIG. 3A at section A-A, and FIG. 3C shows enlarged details of analysis points within the packaging at position P101, ranging from the core through to the packaging skin. In the analysis model, analysis point P101X represents the outer wall surface of the packaging skin and analysis point P101Y represents the inner wall surface of the packaging skin. On the other hand, analysis point P101 represents the core of the biological material and analysis points P101B to P101F represent various locations within the biological material ranging from the core to just immediately the inner wall surface of the packaging skin. Similar analysis point nomenclature is applied at the other analysis positions.

CFD analysis was performed with regard to the thermal properties of the biological material (whole blood), the packaging material (PETG), and the heat exchange fluid (hydrocarbon). The CFD analysis simulated operating conditions of a cryopreservation apparatus including velocity of the heat exchange fluid, temperature of the heat exchange fluid, and flow direction of the heat exchange fluid within the apparatus, in this example, the packaging was assumed to be freely suspended within the apparatus with its leading edge facing the flow direction of the heat exchange fluid as indicated in FIG. 3A.

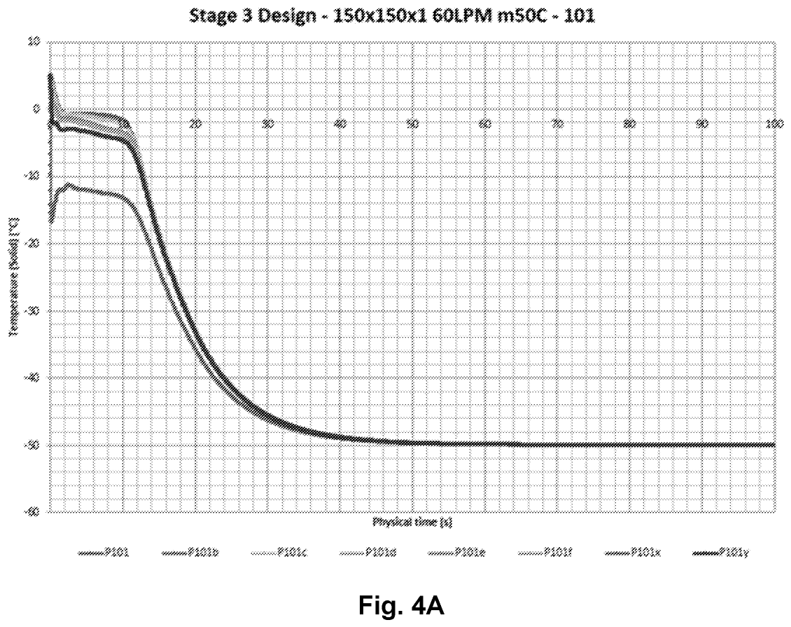

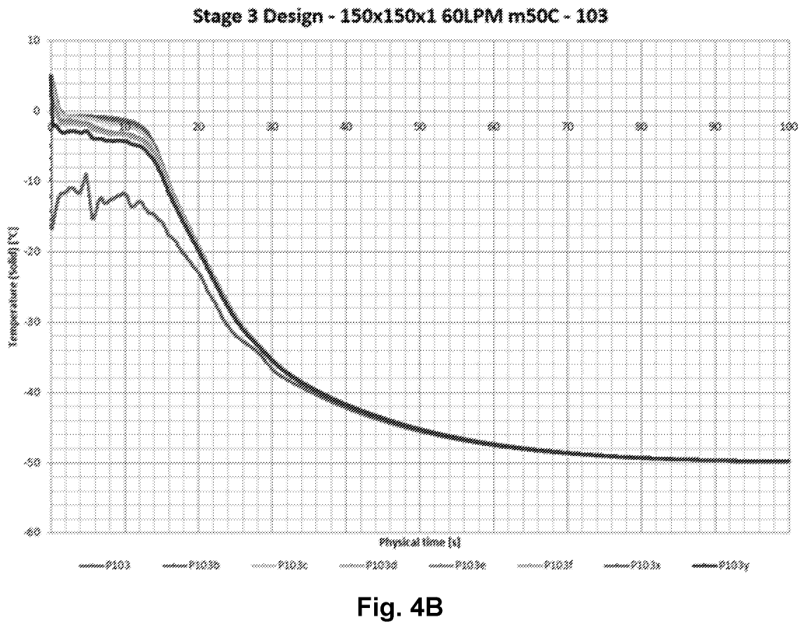

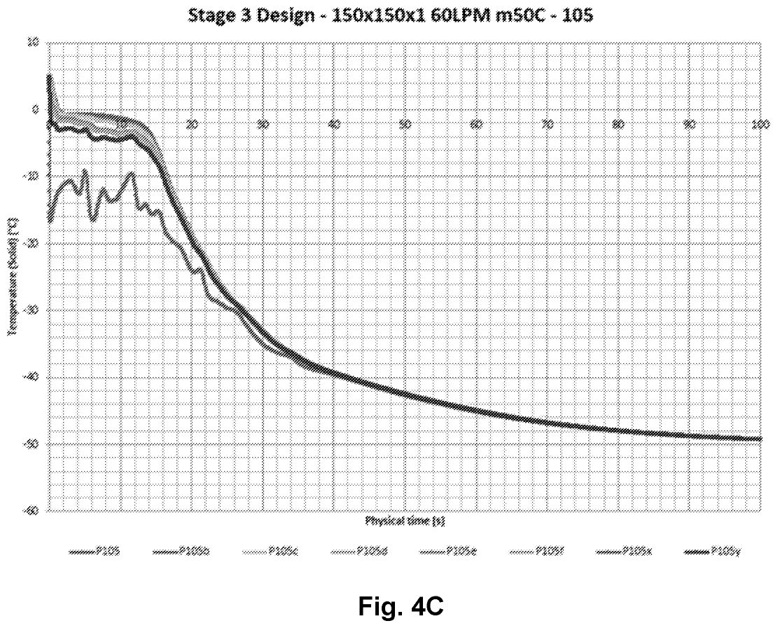

Examples of the CFD analysis results are shown in FIGS. 4A to 4C in the form of plots of temperature gradients over time at the different analysis points at analysis positions P101, P103 and P105. As can be seen from the results, these temperature gradients reflect similar cooling rates at the different analysis positions despite their relative distances from the leading edge of the packaging, demonstrating that the packaging design provides more even heat transfer across the packaging.

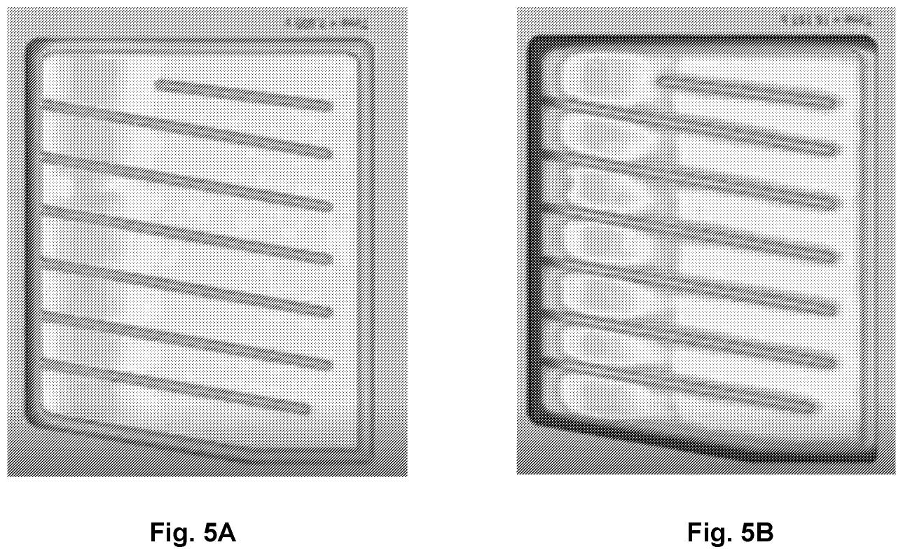

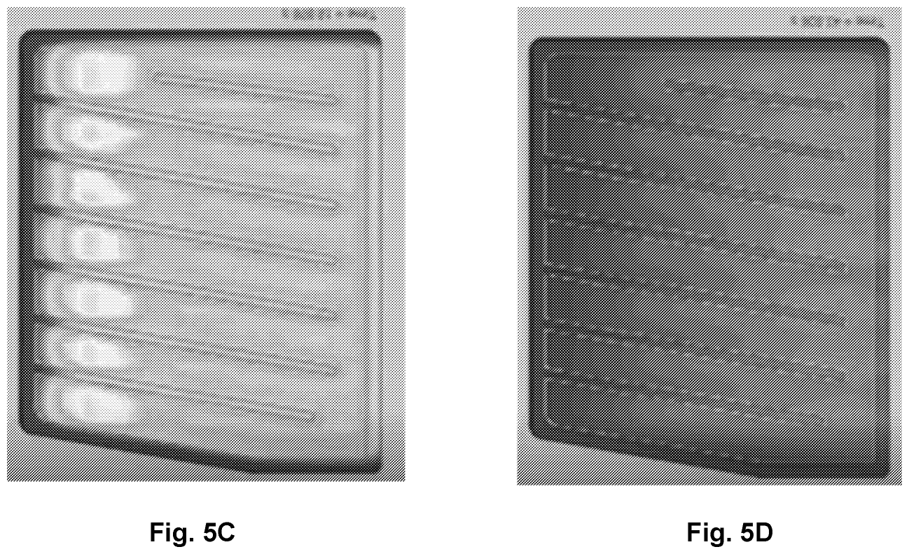

The CFD analysis results are also graphically represented in FIGS. 5A to 5D, which show temperature maps for a central layer of the packaging at subsequent time intervals, and particularly at about 2 s, 17 s, 22 s and 42 s from commencing the flow of the heat exchange fluid during operation of the cryopreservation apparatus, respectively.

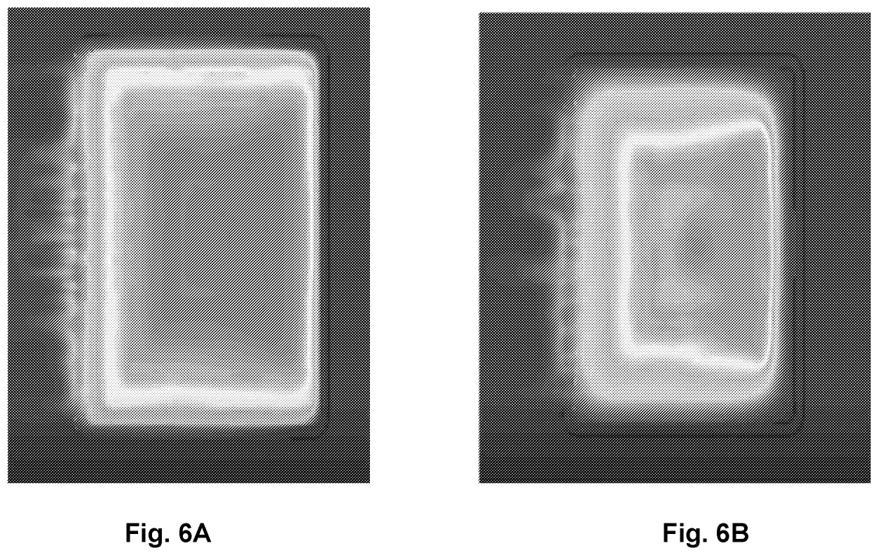

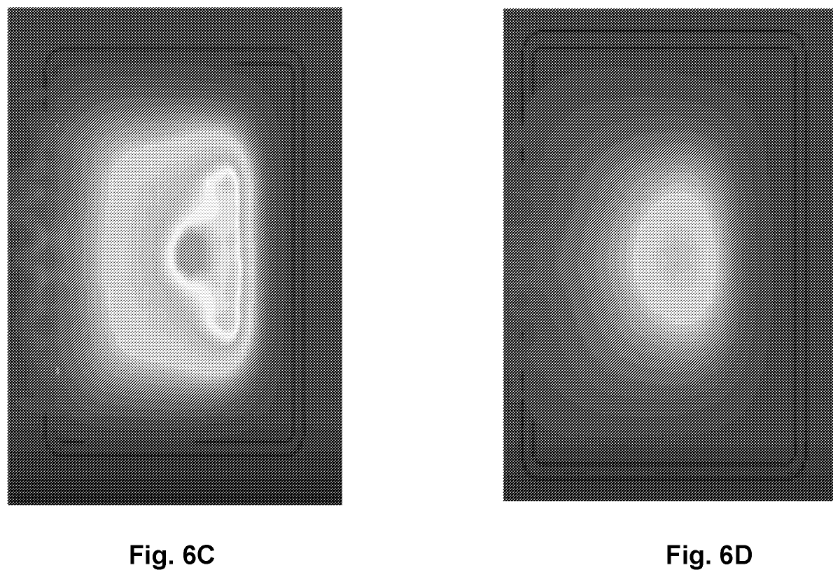

The temperature maps of FIGS. 5A to 5D can be contrasted with temperature maps shown in FIGS. 6A to 6D that have been generated for a similar CFD analysis performed using a model of a conventional IV blood bag for cryopreservation of whole blood in the similar operational conditions.

As can be seen in FIGS. 6A to 6D, the temperature maps for the conventional IV blood bag show significant temperature variation across the bag and a substantial “hot spot” that persists near the centre of the bag as the temperature in the outer regions of the bag are reduced. On the other hand, FIGS. 5A to 5D demonstrate that the design of the packaging in accordance with the present invention substantially reduces this problem by allowing for more even heat transfer within each sub-compartment defined between the thermal contours.

As discussed above, the thermal contours are preferably provided at an angle relative to the leading edge of the packaging, where the angle is selected according to the direction of heat exchange fluid flow in the preservation apparatus. It will be appreciated that CFD analysis can also be used to determine the direction of heat exchange fluid flow for use in selecting the angle of the thermal contours.

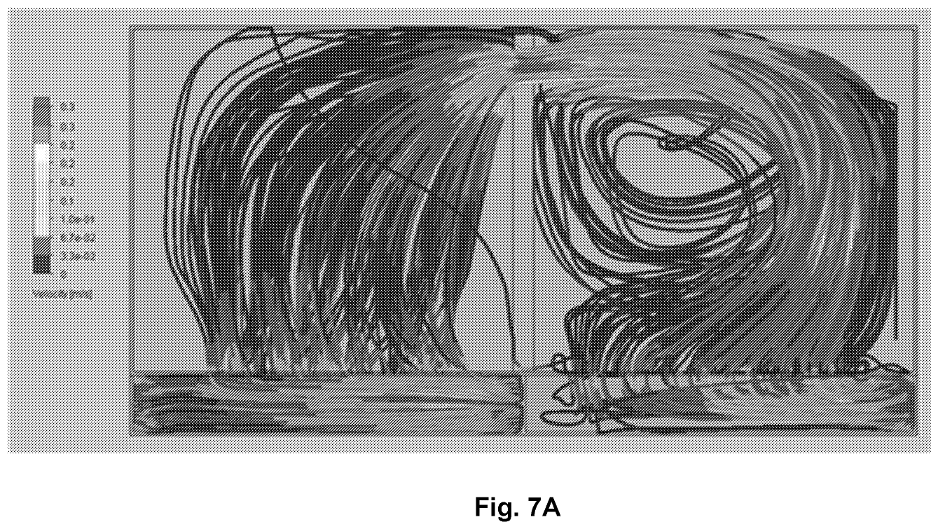

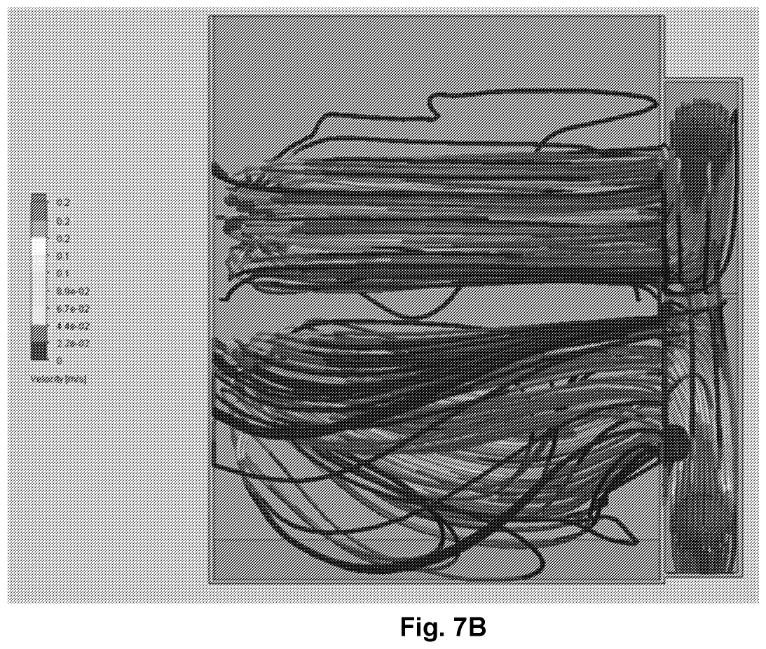

In this regard, FIGS. 7A and 7B show examples of top and side views of CFD analysis results depicting the flow of heat transfer fluid in the cryopreservation apparatus for which the packaging was configured. With reference to FIG. 7A, the packaging is positioned in the left hand side chamber with the leading edge facing downwards into the flow, and it will be appreciated that the angle of the thermal contours will approximately align with the flow direction in this context.

Accordingly, the thermal contours of the packaging may substantially align with the directional flow of the heat exchange fluid in use, to further improve heat transfer between the heat exchange fluid and the biological material in use.

The specific configuration of the thermal contours may be selected with regard to a number of competing design considerations. As discussed above, the thermal contours provide for improved heat transfer by directing heat exchange fluid across the packaging. In preferred embodiments in which the thermal contours define sub-compartments in the packaging, the thermal contours direct fluid flow through each sub-compartment to allow for even heat transfer as demonstrated in the CFD results outline above.

To improve the consistency in heat removal, the thermal contours may be taken to the leading edge to allow for heat transfer fluid to overcome any disruption in flow from raised edging and borders. However, in this embodiment, the thermal contours are not taken to the opposing trailing edge of the packaging, to allow for the contents to be drained out of the bag.

The thermal contours have been included with an angle to allow for directional heat exchange fluid flow as discussed above, but it is noted that the angle of the thermal contours also facilitates draining of the contents from the packaging.

In designing embodiments of the packaging 100, and as a result of validation testing of these embodiments, the applicant also encountered problems when sample volume increases. There was a study conducted which found a correlation between increase sample volume and decrease cell viability. This study specifically focused on the design and manufacture of packaging bags for the preservation of blood and blood products. Validation testing confirmed that as volume increased and bag depth remained the same, cell viability would remain the same.

Table 1 below shows red blood cell comparison testing of increasing packaging bag measurements and associated vitality changes.

TABLE 1
red blood cell comparison testing of increasing packaging
bag measurements and associated vitality changes

| | Bag Size | | | | | | | | | |
|---|---|---|---|---|---|---|---|---|---|---|
| | 30 ml | | | 85 ml | | | | | 140 ml | |
| Volume | 10 ml | 20 ml | 30 ml | 10 ml | 20 ml | 30 ml | 50 ml | 85 ml | 50 ml | 140 ml |
| Ave. Width | 4.2 | 5.8 | 8.4 | 3.7 | 4.0 | 5.9 | 7.3 | 10.8 | 6.3 | 12.2 |
| (mm) | | | | | | | | | | |
| Vitality % | 1.4 | −14.6 | −27.2 | −5.6 | −7.9 | −14.1 | −23.9 | −20.1 | −19.6 | −33.6 |
| Change | | | | | | | | | | |

All blood tested during this experiment was undiluted whole blood with no added cryoprotectant and all cryopreservation and thawing followed standard lab procedures for the apparatus provided for preserving the biological material. The packaging bags were filled and the thickest and thinnest part of the bag was measured using digital callipers. The average cross-sectional width of the bag was estimated, and the trends examined.

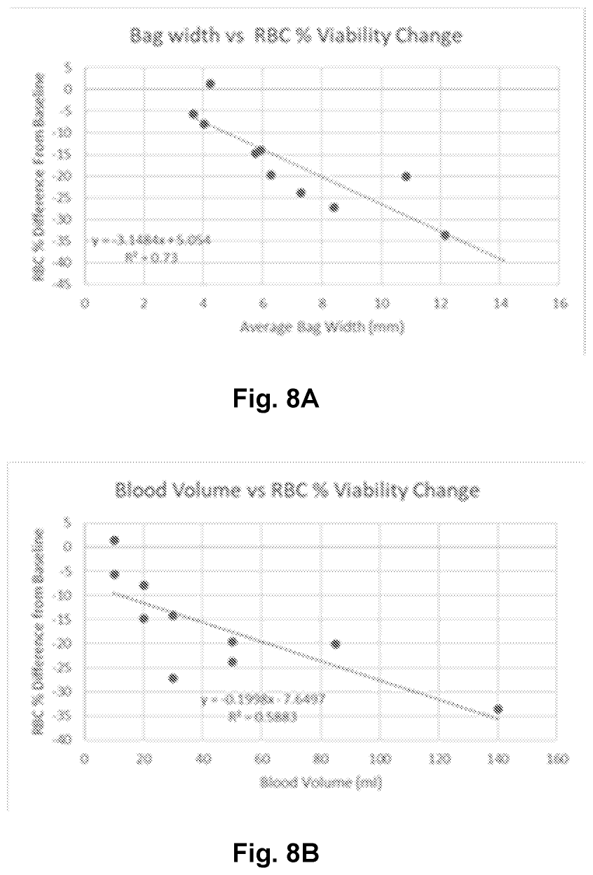

FIGS. 8A and 8B show plots depicting relationships between bag width, blood volume and RBC viability. From the data gathered, it is clear that a trend existed in which increased width of the blood bag caused a decrease in the viability of RBC's.

Based on this trend, the applicant has derived the following linear equation describing the relationship between average packaging apparatus bag width, and the viability of RBC post cryopreservation:

$$RBC \% Difference from Baseline = - 3.1 \; 484 \times Average Bag Width ( mm ) + 5.054 .$$

Using this equation, it is possible to estimate the impact of increasing bag width on the RBC viability, but it is important to note, it is not the only factor effecting the results.

In view of the above design considerations, it will be appreciated that another aspect of the present invention is a method for use in designing packaging for preservation of biological material, wherein, in use, the packaging is filled with biological material and placed in an apparatus for preserving the biological material such that a heat exchange fluid flows around the packaging. In broad terms, the method may include the following steps.

Typically the method will commence with determining a desired packaging geometry including one or more packaging walls configured to define an internal compartment to allow the packaging to be filled with a desired volume of the biological material. The method will also involve determining thermal properties of: the biological material, packaging material for forming the one or more packaging walls, and the heat exchange fluid; and determining operating conditions of the apparatus including: velocity of the heat exchange fluid, temperature of the heat exchange fluid, and flow direction of the heat exchange fluid.

With the packaging geometry, thermal properties and operating conditions determined, the next step of the method will include performing an analysis on the flow of the heat exchange fluid within the apparatus around the filled packaging, in accordance with the determined packaging geometry, thermal properties and operating conditions, to determine expected temperature gradients in the biological material in use.

The expected temperature gradients are used to select a configuration of one or more thermal contours defined across at least one of the packaging walls to improve heat transfer between the heat exchange fluid and the biological material contained in the packaging. The method may subsequently include performing further analysis on the flow of the heat exchange fluid within the apparatus around the filled packaging, including the of one or more thermal contours, in accordance with the determined packaging geometry, the selected configuration of the one or more thermal contours, thermal properties and operating conditions, to determine expected temperature gradients in the biological material in use.

The steps of configuring the thermal contours and performing further analysis thereon may optionally be repeated in an iterative manner until desirable expected temperature gradients are determined.

In one example, this method may be advantageously applied for designing preferred embodiments of the packaging in which the packaging geometry includes opposing first and second walls, the first and second walls being connected together around a substantial portion of their respective perimeters to define the internal compartment, and a plurality of thermal contours defined by the first and second walls being connected together along connection lines, the connection lines being configured to divide the internal compartment into sub-compartments with fluid communication allowed therebetween.

In particular, the method may be extended to include performing the analysis on the flow of the heat exchange fluid within the apparatus around the filled packaging, and using the expected temperature gradients to select a configuration of the thermal contours to provide substantially even heat transfer between the heat exchange fluid and the biological material contained in each of the sub-compartments.

In any event, it will be appreciated that packaging for preservation of biological material as described herein may be provided with thermal contours to facilitate improved heat transfer between the heat exchange fluid and the biological material contained in the packaging, and to particularly avoid problems in conventional packaging which is subject to inconsistent heat transfer resulting in persistent hot spots or the like.

The thermal contours are used to direct the flow of heat exchange fluid across the packaging in a more even manner and therefore provide for more consistent heat transfer. Furthermore, in preferred embodiments, the thermal contours coincide with connections between walls of the packaging which effectively divide the internal compartment of the packaging into sub-compartments, such that the thermal contours can facilitate substantially even heat transfer between the heat exchange fluid and the biological material contained in each of the sub-compartments. In preferred examples, the biological material may be distributed substantially evenly between each of the sub-compartments to further regulate the heat transfer and allow for more consistent results.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

1. Packaging for preservation of biological material, wherein, in use, the packaging is filled with biological material and placed in an apparatus for preserving the biological material such that a heat exchange fluid flows around the packaging, the packaging including:
a) one or more packaging walls configured to define an internal compartment for receiving the biological material; and
b) one or more thermal contours defined across at least one of the packaging walls, wherein, in use, the flow of the heat exchange fluid is at least partially directed by the one or more thermal contours to improve heat transfer between the heat exchange fluid and the biological material contained in the packaging.

2. Packaging according to claim 1, wherein the thermal contours are arranged to substantially align with the flow of the heat exchange fluid in use.

3. Packaging according to claim 1 or claim 2, wherein the packaging includes a plurality of thermal contours that are arranged to be parallel to one another.

4. Packaging according to any one of claims 1 to 3, wherein each of the thermal contours is defined across a respective one of the packaging walls as one of:
a) an elongate depression in the respective packaging wall; and
b) an elongate protrusion in the respective packaging wall.

5. Packaging according to any one of claims 1 to 4, wherein the packaging walls include opposing first and second walls, the first and second walls being connected together around a substantial portion of their respective perimeters to define the internal compartment.

6. Packaging according to claim 5, wherein the one or more thermal contours are defined by the first and second walls being connected together along one or more connection lines, the connection lines being configured to divide the internal compartment into sub-compartments with fluid communication allowed therebetween.

7. Packaging according to claim 6, wherein, in use, the packaging is filled with biological material so that the biological material is distributed between the sub-compartments, and the flow of the heat exchange fluid is at least partially directed by the one or more thermal contours to facilitate substantially even heat transfer between the heat exchange fluid and the biological material contained in each of the sub-compartments.

8. Packaging according to claim 6 or claim 7, wherein the connection lines are configured to ensure that the biological material is distributed substantially evenly between each of the sub-compartments.

9. Packaging according to any one of claims 5 to 8, wherein the first and second walls are connected together along a plurality of edges including a leading edge that faces the flow of heat transfer fluid in use, and an opposing trailing edge, the plurality of thermal contours extending between the leading edge and the trailing edge.

10. Packaging according to claim 9, wherein the trailing edge is substantially parallel to the leading edge.

11. Packaging according to claim 9 or claim 10, wherein at least some of the connection lines interconnect with the leading edge.

12. Packaging according to any one of claims 9 to 11, wherein the thermal contours are arranged at a predetermined angle relative to a direction perpendicular from the leading edge.

13. Packaging according to claim 12, wherein the predetermined angle is selected according to the flow of heat exchange fluid in the apparatus for preserving the biological material.

14. Packaging according to claim 12 or claim 13, wherein the predetermined angle is at least one of:
a) between 0° and 30°;
b) between 5° and 15°; and
c) about 10°.

15. Packaging according to any one of claims 5 to 14, wherein adjacent thermal contours are spaced apart by a predetermined spacing distance.

16. Packaging according to claim 15, wherein the predetermined spacing distance is between 15 mm and 20 mm.

17. Packaging according to claim 15 or claim 16, wherein the predetermined spacing distance is selected to restrict separation of the first and second walls.

18. Packaging according to claim 17, wherein the predetermined spacing distance is selected to restrict separation of the first and second walls to a predetermined separation distance.

19. Packaging according to any one of claims 5 to 18, wherein a packaging depth measured between the first and second walls is at least one of:
a) less than 10 mm;
b) less than 5 mm;
c) between 1 mm and 4 mm; and
d) less than 1 mm.

20. Packaging according to any one of claims 5 to 19, wherein the packaging is configured so that the first and second walls remain substantially parallel to one another when the packaging is filled with the biological material in use.

21. Packaging according to any one of claims 5 to 20, wherein the first and second walls are formed from sheets of a packaging material.

22. Packaging according to claim 21, wherein the packaging material is selected from one of:
a) polymers;
b) polypropylene;
c) polyvinyl chloride;
d) polyethylene terephthalate;
e) ethylene vinyl acetate copolymer;
f) copolymers;
g) ethylene and vinyl acetate;
h) metals;
i) high alloy; and
j) stainless steel.

23. Packaging according to any one of claims 1 to 22, wherein the packaging includes one or more openings for facilitating filling and emptying of the packaging.

24. Packaging according to claim 23, wherein the one or more openings include one or more ports extending through an edge of the packaging.

25. Packaging according to any one of claims 1 to 24, wherein the packaging is configured for preservation of biological material that is selected from one of:
a) whole blood;
b) blood platelets;
c) red blood cells;
d) white blood cells;
e) plasma;
f) b
g) sperm;
h) cells;
i) stem cells;
j) organs or parts thereof; and
k) tissue.

26. Packaging according to any one of claims 1 to 25, wherein the packaging is configured for preservation of biological material to be used for therapeutic treatments.

27. Packaging according to any one of claims 1 to 26, wherein the packaging is configured for at least one of:
a) cryopreservation of biological material;
b) cryostorage of biological material; and
c) thawing of biological material.

28. Packaging according to any one of claims 1 to 27, wherein the packaging is configured for use with a heat transfer rate selected from one of:
a) between 0° C. and 10° C. per minute;
b) between 10° C. and 50° C. per minute;
c) between 50° C. and 100° C. per minute; and
d) greater than 100° C. per minute.

29. Packaging according to any one of claims 1 to 28, wherein the packaging is configured as a bag.

30. Packaging according to any one of claims 1 to 28, wherein the packaging is configured as one of:
a) a straw; and
b) a vial.

31. Packaging for preservation of biological material, wherein, in use, the packaging is filled with biological material and placed in an apparatus for preserving the biological material such that a heat exchange fluid flows around the packaging, the packaging including:
a) opposing first and second walls, the first and second walls being connected together around a substantial portion of their respective perimeters to define an internal compartment; and
b) a plurality of thermal contours defined by the first and second walls being connected together along connection lines, the connection lines being configured to divide the internal compartment into sub-compartments with fluid communication allowed therebetween, wherein, in use, the biological material is distributed between the sub-compartments, and the flow of heat exchange fluid is at least partially directed by the thermal contours to improve heat transfer between the heat exchange fluid and the biological material contained in the sub-compartments of the packaging.

32. A method for use in designing packaging for preservation of biological material, wherein, in use, the packaging is filled with biological material and placed in an apparatus for preserving the biological material such that a heat exchange fluid flows around the packaging, the method including:
a) determining a packaging geometry including one or more packaging walls configured to define an internal compartment to allow the packaging to be filled with a desired volume of the biological material;
b) determining thermal properties of:
i) the biological material;
ii) packaging material for forming the one or more packaging walls; and
iii) the heat exchange fluid;
c) determining operating conditions of the apparatus including:
i) velocity of the heat exchange fluid;
ii) temperature of the heat exchange fluid; and
iii) flow direction of the heat exchange fluid;
d) perform analysis on the flow of the heat exchange fluid within the apparatus around the filled packaging, in accordance with the determined packaging geometry, thermal properties and operating conditions, to determine expected temperature gradients in the biological material in use;
e) using the expected temperature gradients to select a configuration of one or more thermal contours defined across at least one of the packaging walls to improve heat transfer between the heat exchange fluid and the biological material contained in the packaging; and
f) performing further analysis on the flow of the heat exchange fluid within the apparatus around the filled packaging, including the of one or more thermal contours, in accordance with the determined packaging geometry, the selected configuration of the one or more thermal contours, thermal properties and operating conditions, to determine expected temperature gradients in the biological material in use.

33. A method according to claim 32, wherein the method includes repeating steps e) and f) until desirable expected temperature gradients are determined.

34. A method according to claim 33, wherein the packaging geometry includes opposing first and second walls, the first and second walls being connected together around a substantial portion of their respective perimeters to define the internal compartment, and a plurality of thermal contours defined by the first and second walls being connected together along connection lines, the connection lines being configured to divide the internal compartment into sub-compartments with fluid communication allowed therebetween, the method including:
a) performing the analysis on the flow of the heat exchange fluid within the apparatus around the filled packaging; and
b) using the expected temperature gradients to select a configuration of the thermal contours to provide substantially even heat transfer between the heat exchange fluid and the biological material contained in each of the sub-compartments.
